(19) United States
(12) Patent Application Publication
(10) Pub. No.: US 2008/0175461 A1
(43) Pub. Date: Jul. 24, 2008

(54) Method for displaying images by means of a graphics user interface of a digital image information system

(76) Inventor: Sven Hentschel, Bennewitz (DE)

Correspondence Address: HARNESS, DICKEY & PIERCE, P.L.C., P.O.BOX 8910, RESTON, VA, 20195, US

(21) Appl. No.: 12/003,303

(22) Filed: Dec. 21, 2007

(30) Foreign Application Priority Data
Dec. 28, 2006 (DE) 10 2006 061 888.2

(51) Int. Cl. A61B 5/00

(52) U.S. Cl. 382/131

(57) ABSTRACT

A method is disclosed for displaying images by way of a graphics user interface of a digital image information system. In at least one embodiment, a two-dimensional image detail which contains the marked image feature is displayed in the same segment as the three-dimensionally displayed image of the graphics user interface on the basis of an arbitrary user input for marking an image feature of an image which is displayed three-dimensionally in a segment of the graphics user interface.

### PRIORITY STATEMENT

The present application hereby claims priority under 35 U.S.C. §119 on German patent application number DE 10 2006 061 888.2 filed Dec. 28, 2006, the entire contents of which is hereby incorporated herein by reference.

### FIELD

Embodiments of the invention generally relate to the technical field of digital image information systems. They may relate in particular to a method for displaying images by way of a graphics user interface of a digital image information system, to an electronic logic device for controlling the display of images by way of a graphics user interface of a digital image information system, to a machine-legible program code which contains control commands which cause the logic device to carry out the method according to an embodiment the invention, and/or to a storage medium for the program code.

### BACKGROUND

Nowadays, medical image information systems are increasingly being used in clinics and doctors' practices for image object management, specifically for storage, archiving and provision of medical image objects. “PACS systems” (PACS=Picture Archiving and Communications System) which were originally used purely for the purpose of image data management have nowadays generally become fused with administratively oriented information systems, such as radiology information systems (RIS) or hospital information systems (HIS), to form integrated image information systems. In medical image information systems, medical images produced by so-called imaging modalities, for example computing-tomography scans, magnetic resonance imaging scans, positron emission tomography scans, and angiographs and monographs, are sent in the form of pixel data via a communication network to an image storage and image archiving system, where they are stored and archived together with administrative text data, such as the patient's name, date of birth, patient number, equipment number, examination date, study number and much more. The administrative text data is normally stored in a separate database, which is associated with respective storage devices for storing the extensive pixel data of the image objects.

The pixel data for the image objects that are produced is stored in a typical manner first of all in a short-term data store, for example a non-volatile store with a plurality of coupled hard discs, a so-called RAID store (RAID=redundant array of independent discs). From there, it can be retrieved from screen workstations within a very short time, for example within a few seconds, for assessment, analysis or processing. Once a variable time period has elapsed, for example six to twelve months or after assessment, the pixel data is transferred to a long-term data store for permanent archiving, with this store, for example, being in the form of a so-called juke box with a plurality of tape stores or magneto-optic or optical discs, such as CDs (compact discs) or DVDs (digital versatile discs). Because of the legal regulations, the image data has to be stored, and must be capable of being displayed, for a longer time period, for example of ten to thirty years.

The image objects that are produced are stored in a data format that may be chosen by the manufacturers in a medical image information system. Since various components of a medical image information system are often produced by different manufacturers, an open standard, which is referred to as DICOM (Digital Imaging and Communication) has been created in order to harmonize different data formats, in conjunction with the American College of Radiology and the National Electrical Manufacturers Association and, inter alia, this standard defines the structure of the data formats and of the descriptive parameters for radiological images, the commands for interchanging these images, such as the description of other data objects, such as image sequences, examination series and assessments. According to DICOM, an image object includes a header component (text data component) in which administrative information, such as the patient's name, the date of birth, the patient number and the like are included, and a pixel component (pixel data), in which the pixel-based image content of the image recorded by an imaging modality is included.

The pixel-based image contents of the image objects are displayed by way of graphics user interfaces (screens) in the form of two-dimensional (2D) slice images or in the form of three-dimensional (3D) images (volume displays) which are determined by computer from the two-dimensional slice images. In addition to impressive visualization of anatomical volumes, the 3D images provide the viewer with a quick overview of the position of the two-dimensional slice images. However, practical experience indicates that 3D images are used fairly rarely for diagnosis by medical practitioners. In fact, the doctors, in particular radiologists, base their diagnosis in a familiar manner on the two-dimensional slice images and in fact consider 3D images just as an additional aid without any significant value for assessment.

Irrespective of this, volume displays are frequently used for demonstration purposes or as snap shots for referring doctors and for operation planning. The images are normally displayed on a graphics user interface, such as that provided by a screen workstation, in such a way that different displays of the desired images are displayed in a plurality of mutually independent display areas, which do not overlap one another, of the graphics user interface, so-called segments. For example, a computer-determined 3D image is displayed in this way in one segment, while selectable two-dimensional slice images of the 3D image can be displayed in segments other than this, for example in the form of sagittal, frontal or transverse slices.

This has the particular disadvantage that an image display of this type necessarily limits the segment size, because of the screen size, and this may have an adverse effect on the capability to identify image features.

Until now, a joint display of volume displays and associated slice images has been produced such that an appropriate slice image can be displayed in another segment by optional placing of a slice plane which entirely subdivides the volume display. By way of example, in a volume display of the lungs, a frontal slice plane may be placed at a specific image depth such that the two individual lungs are displayed in a desired frontal section.

The primary disadvantage in this case is the fact that the generation of the desired slice image on the basis of the normally very large amount of data in the slice plane that completely cuts through the volume display is extremely time-consuming and computation-intensive. Furthermore, it is relatively tedious for the user to have to place the slice plane such that the desired image feature is displayed such that it can be seen well in the resultant two-dimensional slice image.

### SUMMARY

In contrast, at least one embodiment of the present invention provides a method displaying images by way of a graphics user interface, which makes it possible to reduce or even avoid at least one of the stated disadvantages.

M. Jahnke “3D-Exploration von Volumendaten” [3D exploration of volume data], Thesis, Rhein, Friedrich-Wilhelms-Univ., Bonn 1998, Institute for Information Technology, specialist computer graphics, the entire contents of which is hereby incorporated herein by reference, discloses various procedures for displaying images by way of a graphics user interface, in each of which a magic lens (probe) is used for a 3D display, with the magic lens being used for marking and other types of display of a part of the 3D display that is located within the magic lens.

At least one embodiment of the invention discloses a method for displaying images by way of a graphics user interface of a digital image information system.

In this case and in the following text, the expression “images” is intended to mean pixel components of image objects with the latter, as already explained initially, comprising a text component containing administrative information and a pixel component containing the pure image data. The text component contains an identifier “key” associated with the pixel component, in order to identify the pixel data of an image object. The identifier may be part of the administrative information, or may be newly generated. The image objects may each contain one or more images, for example a sequence of images, produced by an imaging modality. Furthermore, the image objects may each contain images which have been produced from a plurality of imaging modalities.

The digital image information system managed by an electronic data management device comprises at least one data storage device, such as a long-term store and/or a short-term store, in which the pixel components of the image objects are stored, for archiving and storage of text and pixel components of image objects. The long-term and short-term stores are in the form of non-volatile data stores, for example in the form of an RAID hard-disc store, a tape or disc store, in particular a juke box with a plurality of magneto-optical or optical discs, such as CDs or DVDs. Furthermore, a database associated with the data storage devices is provided, in which the text components of the image objects are stored.

A display of the image objects, in particular of the pixel data of the image objects, is provided by at least one graphics user interface, which can be made available by a screen workstation. The display of the image objects, in particular of the pixel-based image data, is controlled by a logic device which may be part of the data management device for management of the digital image information system.

The method according to at least one embodiment of the invention is essentially distinguished in that, in an image which is displayed three-dimensionally in a segment of the graphics user interface, a two-dimensional image detail (slice image) which contains the marked image feature is displayed in the same segment as the three-dimensionally displayed image of the graphics user interface on the basis of an arbitrary user input (user interaction), which is used to mark a displayed image feature. By way of example, an image feature such as this is an anatomical structure in an anatomical volume display.

As already explained initially, the segment of the graphics user interface is a restricted area of the screen which is used to display a two-dimensional or three-dimensional image. A screen may normally comprise one or more segments, so that a plurality of images can be displayed at the same time.

The method according to at least one embodiment of the invention advantageously makes it possible to display an image feature, which is of interest to the viewer and is displayed in the form of a volume display, in a two-dimensional slice image which corresponds to a detail of the volume display. In contrast to the conventional display form explained initially, the display of the image feature of interest in a two-dimensional detail of the volume display requires the processing of relatively small amounts of data, so that the display can be produced in a relatively short time, with comparatively little computation power. Furthermore, by manual interaction, the user can very easily mark the image feature of interest, and cause it to be displayed. In addition, it is advantageous for the two-dimensional image detail which contains the desired image feature to be displayed in the same segment as the volume display, so that the image feature of interest can be viewed in conjunction with the volume display.

The two-dimensional detail of the volume display containing the image feature of interest is displayed in such a way that it does not cover the marked image feature in the volume display, and is thus placed alongside the volume display of the image feature.

Conventional marking methods based on standard routines, such as so-called 3D picking, can be used to mark the image feature to be displayed in the volume display, making it possible to obtain the depth information of the image feature of interest in order to create a slice plane containing the image feature by defining a screen position by user interaction.

The size and/or shape of the two-dimensional image detail can be set as required on the basis of a user input. Alternatively, it would also be possible to provide a predetermined size and/or shape of the two-dimensional image detail.

At least one embodiment of the invention also extends to a logic device for the digital image information system, which is provided with a machine-legible program code (computer program) which includes control commands that cause the logic device to carry out the method according to at least one embodiment of the invention, as described above.

Furthermore, at least one embodiment of the invention extends to a machine-legible program code (computer program) for the logic device for the digital image information system, which contains control commands which cause the electronic logic device to carry out the method according to at least one embodiment of the invention, as described above.

Furthermore, at least one embodiment of the invention extends to a storage medium (computer program product) with a machine-legible program code stored in it for a logic device for the digital image information system, which contains control commands which cause the logic device to carry out the method according to at least one embodiment of the invention, as described above.

### BRIEF DESCRIPTION OF THE DRAWINGS

The invention will now be explained in more detail using example embodiments, with reference to the attached drawings, in which:

FIG. 1 shows a first example embodiment of the method according to the invention, showing a volume display of human anatomical structures and a two-dimensional image detail superimposed;

FIG. 2 shows a second example embodiment of the method according to the invention, in which a volume display of human anatomical structures and a two-dimensional image detail are arranged alongside one another;

FIG. 3 shows a schematic illustration, in order to show the 3D picking method.

### DETAILED DESCRIPTION OF THE EXAMPLE EMBODIMENTS

The terminology used herein is for the purpose of describing particular embodiments only and is not intended to be limiting of the present invention. As used herein, the singular forms “a”, “an”, and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described. Like numbers refer to like elements throughout. As used herein, the terms “and/or” and “at least one of” include any and all combinations of one or more of the associated listed items.

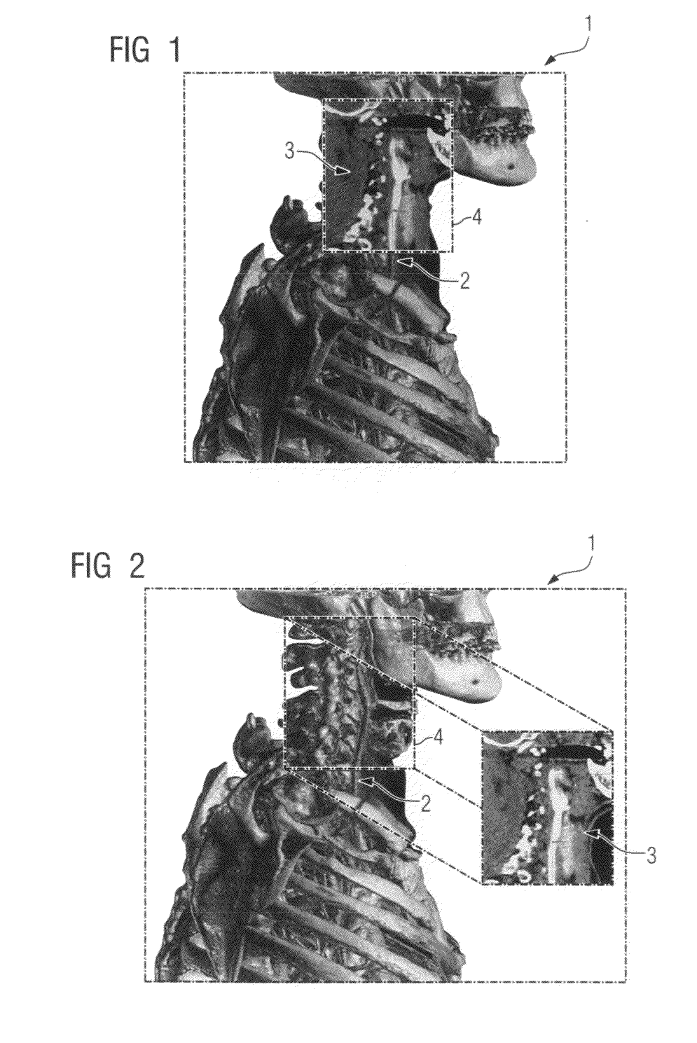

FIG. 1 will be considered first of all, illustrating a first example embodiment of the method according to the invention.

FIG. 1 shows a part of the screen of a graphics user interface in which a volume display of human anatomical structures in the head/thorax area is illustrated. The graphics user interface is part of a digital image information system, as already explained further above, for example a PACS system. In order to avoid unnecessary replication, reference is made to the statements there. The volume display is produced in a segment of the graphics user interface that is annotated with the reference number 1 overall and is set up in a part of the screen of the graphics user interface.

Let us assume that the image viewer is interested in a specific (arbitrary) anatomical image feature, in this case by way of example, the right-hand aorta carotis 2 of the patient.

For this purpose, the viewer marks the screen position corresponding to the aorta carotis 2, for example by a mouse click. The aorta carotis 2 is identified as an image feature of interest, for example by way of a 3D picking method.

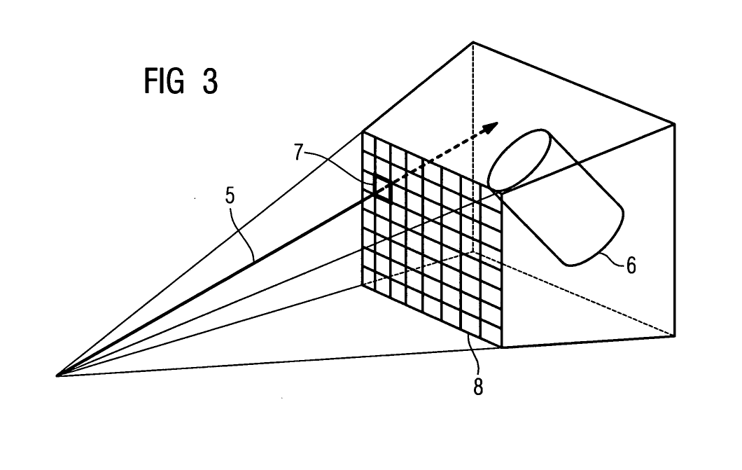

The 3D picking method is a conventional method, which is well known per se by those skilled in the art, for identification of a desired image feature at a marked screen position. As shown in FIG. 3, “ray” 5 is passed though the volume of the volume display, for example at the screen position 7, marked by the user, on a screen 8 which is subdivided into various pixels and adds up the opacities of the individual voxels in the volume until a threshold value is exceeded. The process stops at this voxel point and the depth information obtained in this way is used to produce the three-dimensional coordinate of the image feature 6 corresponding to the marked screen position 7. The threshold value is calculated from the display parameters of the projection of the volume on the screen 8.

3D picking is therefore used to find the three-dimensional coordinate of the desired image feature 6 in the volume display and to identify the corresponding two-dimensional slice for the “picked” image feature 6.

A two-dimensional slice image 3 of the marked image feature 6, in this case the right-hand aorta carotis 2, is then displayed in the segment 1 on the screen. The two-dimensional slice image 3 corresponds just to a detail from the volume display, which is bounded by the detail frame 4. The detail frame 4 and therefore the size and shape of the two-dimensional slice image 3 can be predetermined arbitrarily by user interaction, such as mouse operation. It will be just as possible to preset the detail frame 4.

In FIG. 1 the two-dimensional slice image 3 is partially superimposed on the image feature shown in the volume display, so that part of the aorta carotis 2 is covered.

In contrast FIG. 2 shows a different display form, in which the two-dimensional slice image 3 is arranged alongside the image feature shown in the volume display.

The method according to an embodiment of the invention therefore makes it possible to obtain more information than from conventional joint displays of 3D and 2D images. Furthermore, the image features of interest can be marked in a simple manner by user interaction, and can be displayed quickly and with comparatively little computation power in an image detail in the form of a two-dimensional slice image. The two-dimensional slice image, which can be produced by user interaction, of the image feature of interest in the volume display may be regarded as a “volume lens”, and in this case the image feature of interest may be displayed with or without magnification.

The method according to an embodiment of the invention can be employed usefully in many applications, such as:

1) CT (computed tomography) in angiology, with the volume display being based on a CT VRT (computer-tomography volume rendering technique) or CT MIP (computer-tomography maximum intensity projection) technique, and with the volume lens showing a CT MPR (computer-tomography multi-planar reformatting).
2) CT (computed tomography) in cardiology, with the volume display being based on a CT VRT (computer-tomography volume rendering technique) or CT MIP (computer-tomography maximum intensity projection) technique, and with the volume lens showing a CT MPR+τ film (computer-tomography multi-planar reformatting).
3) MRI (magnetic resonance imaging) in angiology, with the volume display being based on a CT VRT (computer-tomography volume rendering technique) or CT MIP (computer-tomography maximum intensity projection) technique, and with the volume lens showing MR MPR (magnetic resonance imaging multi-planar reformatting).
4) PET/CT (positron emission tomography/computed tomography) in oncology, with the volume display being based on a CT (computed tomography) linked VRT (volume rendering technique) or PET MIP (positron emission tomography maximum intensity projection) technique, and with the volume lens showing a hybrid display MPR (multi-planar reformatting) of the dynamic CT volume.
5) CT (computed tomography) in oncology with the volume display being based on a CT VRT (computer-tomography volume rendering technique) or MIP (maximum intensity projection) technique, and the volume lens showing a hybrid display MPR (multi-planar reformatting) of the native, venous etc. volume.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDS; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for displaying images by way of a graphics user interface of a digital image information system, the method comprising:
marking an image feature of an image via an input and marking element of a user, displayed three-dimensionally in a segment of the graphics user interface, to determine depth information of the image feature;
identifying a two-dimensional layer containing the marked image feature based upon the depth information; and
displaying a slice image in the same segment as the three-dimensionally displayed image of the graphics user interface, displayed in a form of a two-dimensional image detail obtained by restricting the two-dimensional slice by a detail frame, the detail frame being at least one of predetermined and preset via user input by the input and marking element.

2. The method as claimed in claim 1, wherein the displayed two-dimensional image detail covers the marked image feature.

3. The method as claimed in claim 1, wherein the two-dimensional image detail is displayed alongside the marked image feature.

4. The method as claimed in claim 1, wherein the image feature is marked on the basis of a user input via a 3D-picking method.

5. A logic device for controlling the display of images by way of a graphics user interface of a digital image information system, provided with a program code which, when executed, contains control commands which cause the logic device to carry out the method as claimed in claim 1.

6. A machine-legible program code for a logic device as claimed in claim 4 for controlling the display of-images by way of a graphics user interface of a digital image information system.

7. A storage medium comprising the machine-legible program code as claimed in claim 6.

8. The method as claimed in claim 2, wherein the two-dimensional image detail is displayed alongside the marked image feature.

9. The method as claimed in claim 2, wherein the image feature is marked on the basis of a user input via a 3D-picking method.

10. The method as claimed in claim 3, wherein the image feature is marked on the basis of a user input via a 3D-picking method.

11. A computer readable medium including program segments for, when executed on a computer device, causing the computer device to implement the method of claim 1.

12. A computer readable medium including program segments for, when executed on a computer device, causing the computer device to implement the method of claim 2.

13. A computer readable medium including program segments for, when executed on a computer device, causing the computer device to implement the method of claim 3.

14. A computer readable medium including program segments for, when executed on a computer device, causing the computer device to implement the method of claim 4.

15. A logic device for controlling the display of images by way of a graphics user interface of a digital image information system, comprising the computer readable medium of claim 11.

16. A logic device for controlling the display of images by way of a graphics user interface of a digital image information system, comprising the computer readable medium of claim 12.

17. A logic device for controlling the display of images by way of a graphics user interface of a digital image information system, comprising the computer readable medium of claim 13.

18. A logic device for controlling the display of images by way of a graphics user interface of a digital image information system, comprising the computer readable medium of claim 14.
